(19) United States
(12) Patent Application Publication
(10) Pub. No.: US 2003/0166237 A1
(43) Pub. Date: Sep. 4, 2003

(54) Antifouling paint composition comprising rosin and enzyme

(76) Inventors: Knud Allermann, Rungsted Kyst (DK); Ib Schneider, Copenhagen (DK)

Correspondence Address: BROWDY AND NEIMARK, P.L.L.C., 624 NINTH STREET, NW, SUITE 300, WASHINGTON, DC, 20001-5303, US

(21) Appl. No.: 10/239,650

(22) PCT Filed: Mar. 23, 2001
(86) PCT No.: PCT/DK01/00202

(30) Foreign Application Priority Data
Mar. 24, 2000 (DK) PA 2000 00506

(51) Int. Cl. C12N 9/32; C12N 9/24; C12N 9/56; C09D 5/16

(52) U.S. Cl. 435/204; 435/222; 435/200; 106/16

(57) ABSTRACT

An antifouling paint composition comprising an enzyme, such as endopeptidase, Subtilisin (EC 3.4.21.62) and Alcalase®, and a rosin compound, wherein the enzyme is effective to reduce or prevent fouling by aquatic organisms of a surface coated with the composition. Also disclosed is a method for preventing fouling of a surface by aquatic organisms.

### FIELD OF THE INVENTION

The present invention relates to the field of preventing or reducing fouling of surfaces of structures that are occasionally or continuously immersed in water such as ship hulls and marine structures. More specifically, there is provided an antifouling paint composition comprising an enzyme and a rosin compound that is effective in respect of inhibiting the attachment and settlement of aquatic organisms, in particular barnacles.

### TECHNICAL BACKGROUND AND PRIOR ART

All surfaces in aquatic environments are subject to intense fouling pressure by bacteria, protozoa, algae and invertebrates. This process is called fouling The control of fouling is of particular concern to marine shipping operations and marine engineering (offshore constructions, heat exchangers, marine sensors, water inlets, aquaculture constructions etc.). Fouling on the hulls of ships for example increases frictional drag with a corresponding decrease in speed and manoeuvrability and an increase in fuel consumption and increased maintenance costs associated with removal of the fouling. Furthermore, even a small number of organisms attaching themselves to the propellers of a ship can significantly reduce the propellers' efficiency or create corrosion problems.

An important group of marine organisms that contributes significantly to the fouling process is the group of crustacean organisms that are commonly referred to as barnacles. These organisms belong to the Cirripedia subclass of the order Crustacea. A common feature for Cirripedia is that the adult stages are sessile and become attached to solid surfaces by the secretions of a cement gland on their first antenna. The Cirripedia subclass includes four orders: Thoracica; Acrothoracica, Ascothorica and Rhizocephalae. Of these, organisms of Thoracica that belongs to the genus Balanus, also referred to as acorn shell or rock barnacles, are commonly involved in fouling of submerged surfaces such as ship hulls,

Presently, the majority of antifouling paint compositions contain a toxic substance, such as heavy metals, which slowly reacts with e.g. sea-water to give a salt soluble in water and which is leached from the matrix of the paint. However, the steady accumulation of these toxic substances in the marine environment has adversely affected marine life. Thus, such toxic substances impose a world-wide pollution risk to the environment and therefore restrictions have been or are being applied to their use and many of them have already been banned in many countries. Additionally, most of the presently known antifouling paint compositions are based on synthetic binder components which can impose a serious health risk to people such as painters working with the paint compositions on a daily basis.

Accordingly, there is a need for antifouling methods and compositions that do not use toxic additives or binders in such a way as to substantially harm the environment or impose a health hazard to humans. This need has i.a. resulted in attempts to develop alternative environmentally-friendly and non-polluting antifouling methods to overcome the above problems, including the use of enzymes.

Thus, U.S. Pat. No. 5,998,200 describes a method for preventing fouling of an aquatic apparatus in contact with an aquatic environment by an aquatic organism, by applying a composition containing an inert matrix having an enzyme chemically bonded thereto. The matrix or binder is preferably a polyurethane polymer such as hydrophilic polyurethane prepolymers, and the chemically bonded enzyme, such as a protease, is capable of hindering attachment of aquatic organisms such as bacteria, fungi, algae, arthropods and molluscs.

U.S. Pat. No. 5,770,188 describes an antifouling paint composition which comprises a lipid-coated enzyme showing high activity in organic solvents as a result of coating with a lipid having 6 to 30 carbon atoms, and a paint resin. It is described that paint resins for organic solvent paints and water paints are applicable.

U.S. Pat. No. 5,919,689 discloses a marine antifouling composition or paint which comprises base materials, such as epoxy, polyurethane, polyester, fiberglass, silicone, or acrylic materials, and amylolytic or proteolytic enzymes and micro-organisms which produce amylolytic or proteolytic enzymes, where the enzymes and the micro-organism result in a reduction or prevention of fouling of marine surfaces coated with the composition.

However, none of the prior art methods or paint compositions known to the present inventors disclose or suggest the use of a combination of an enzyme and a rosin compound of natural origin in an antifouling paint composition for reducing or preventing fouling of surfaces such as marine surfaces

According to the present invention, there is now provided an antifouling paint composition which comprises at least one enzyme and at least one rosin compound wherein the enzyme is present in an amount that is effective with respect to reducing or preventing fouling of the surface coated with said composition, wherein the rosin compound is of natural origin. Additionally, by using a rosin compound of natural origin as binder, the antifouling paint composition of the present invention is highly advantageous with respect to health hazards as compared to synthetic binders.

### SUMMARY OF THE INVENTION

Accordingly, the invention relates in one aspect to an antifouling paint composition which comprises at least one enzyme and at least one rosin compound wherein the enzyme is present in an effective amount to reduce or prevent fouling of a surface coated with said composition.

There is also provided a method for preventing fouling of a surface by an aquatic organism, by applying to the surface an effective amount of an antifouling paint composition according to the invention.

In a further aspect there is provided an antifouling paint composition which comprises at least one subtilisin (EC 3.4.21.62) said subtilisin having the following characteristics: (i) optimum activity at a pH in the range of about 7-10, and (ii) optimum activity at a temperature in the range of about 55-65° C.

In still further aspects the invention relates to the use of subtilisin (EC 3.4.21.62) in an antifouling paint composition said subtilisin having the following characteristica: (i) optimum activity at pH in the range of about 7-10, and (ii) optimum activity at a temperature in the range of about 55-65° C.

### DETAILED DISCLOSURE OF THE INVENTION

The primary objective of the present invention is to provide an antifouling paint composition. Thus, there is provided a composition which effectively reduce or prevent fouling of marine surfaces coated with the composition according to invention.

The antifouling composition according to the invention is useful in aqueous environments such as fresh, salt or brackish water, including cooling tower systems, fresh water piping systems, salt water piping systems, ponds, lakes, harbours and desalination systems.

The term “fouling” is used herein to designate the attachment of aquatic organisms to the surfaces of structures occasionally or permanently submerged in an aqueous environment, such as bacteria, protozoa, algae and invertebrates including barnacles and mussels.

The antifouling paint composition according to the invention comprises at least one enzyme and at least one rosin compound wherein the enzyme is present in an effective amount to reduce or prevent fouling of a surface coated with the composition.

In one aspect of the invention the at least one enzyme is selected from the group consisting of a proteolytically, hemicellulolytically, a cellulolytically, a lipolytically and an amylolytically active enzymes.

In the present context “proteolytically active” relates to any enzyme having the capability to degrade proteins. “Hemicellulolytically active” relates to any enzyme such as xylanases, having the capability to degrade at least one substance belonging to the group of compounds generally referred to as hemicellulose including xylans and mannans such as Endo-1,4beta-xylanase (E.C. 3.2.1.8), Xylan endo-1,3-beta-xylosidase (E.C. 3.2.1.32) Glucuronoarabinoxylan endo1,4beta-xylanase (E.C. 3.2.1.136), Beta-mannosidase (E.C. 3.2.1.25), Mannan endo-1,4beta-mannosidase (E.C. 3.2.1.78) and Mannan endo-1,6-beta-mannosidase (E.C. 3.2.1.101).

Enzymes having “cellulolytic activity” are also generally referred to as cellulases and is used herein to designate any cellulose hydrolysing enzyme.

“Lipolytically active” enzymes are also generally referred to as lipases and are used herein to designate any triacylglycerol hydrolysing enzyme, including such enzymes that are capable, of splitting of fatty acids having short, medium and long chain lengths. Other enzymes having lipolytic activity which are encompassed by the present invention include phospholipases, lysophospholipases, acylglycerol lipases and galactolipases.

“Amylolytically active” enzymes includes, in the present context, amylases, such as α- and β-amylases, amyloglucosidases, pullulanases, α-1,6-endoglucanases, α-1,4-exoglucanases and isoamylases.

In a one aspect of the invention the at least one enzyme is a protease including an endopeptidase such as the endopeptidase Subtilisin (EC 3.4.21.62).

The beneficial antifouling effect of the protease is believed to be due to the capability of the protease to degrade proteinaceous materials secreted by e.g. barnacles as adhesives for settlement.

In accordance with the invention the endopeptidase Subtilisin (EC 3.4.21.62) can advantageously be used by applying a commercially available enzyme preparation such as Alcalase®. In a presently preferred embodiment the enzyme preparation Alcalase 2.5 L, Type DX® is applied. However it is also contemplated that other Alcalase® products, including Alcalase 2.0 T®, Alcalase 3.0 T® and Alcalase 2.5 L, Type DX®, can be applied in accordance with the present invention. Such Alcalase® enzyme preparations are available from Novozymes (Novozymes, Novo Allé, 2880 Bagsvaerd, Denmark). Alcalase® is a serine-type protease characterised by a good performance at elevated temperatures and moderate alkalinity. Further information with respect to e.g. activity characteristics of the various Alcalase-products is described in the product sheet from Novozyme A/S (B259-GB).

However, it is also within the scope of the invention that other proteases having essentially the same characteristics as the protease of Alcalase® can be successfully applied in accordance with the invention. Thus, it is contemplated that other proteases, such as subtilisins, having essentially the same temperature and pH profiles as the Alcalase, can be utilised. The temperature and pH profiles of the Alcalase can, be found on the product sheet from Novozyme A/S (B259f-GB). Accordingly, it is with the scope of the invention that a subtilisin (EC 3.4.21.62) having the following characteristica: (i) optimum activity at a pH in the range of about 7-10, and (ii) optimum activity at a temperature in the range of about 55-65° C., may advantageously be applied.

Additionally, it is also with in the scope of the invention that more than one protease can be applied, e.g. by the use of complex enzyme preparations comprising several proteases.

As it is mentioned above, an important component of the antifouling paint composition according to the invention is a rosin compound. Rosin is a solid material that e.g. occurs naturally in the oleo rosin of pine trees and is typically derived from the oleo resinous exudate of the living tree, from aged stumps and from tall oil produced as a by-product of kraft paper manufacture.

Rosin compounds have a number of highly desirable properties for use as binders in antifouling paints such as e.g. being fairly non-toxic to humans, being compatible with a large number of other binders and being relatively inexpensive and readily available from natural resources.

Thus, rosins are used in paints as binders, and thereby provide a rather non-toxic alternative to synthetic and more toxic binders such as e.g. polymeric binder components as epoxy, polyvinylacetate, polyvinylbutyrate and polyvinylchioride acetate.

Rosin is typically classed as gum rosin, wood rosin, or as tall oil rosin which indicates its source. The rosin materials can be used unmodified, in the form of esters of polyhydric alcohols, in the form of rosins polymerised through the inherent unsaturation of the molecules or in the form of hydrogenated rosin. Thus, rosin can be further treated by e.g. hydrogenation, dehydrogenation, polymerisation, esterification, and other post treatment processes. Additionally, rosin with e.g. free carboxylic acid groups are capable of reacting with metals and thereby forming rosin metal salts.

Accordingly, the rosin compound of the antifouling paint composition of the present invention is at least one selected from rosins, rosin derivatives, and rosin metal salts. Examples of rosins include tall rosin, gum rosin, and wood rosin. Examples of rosin derivatives include hydrogenated rosins, modified rosins obtained by reacting rosins with maleic anhydride, formylated rosins, and polymerised rosins. Examples of rosin metal salts include zinc rosinates, calcium rosinates, copper rosinates, magnesium rosinates, and products of the reaction of rosins with compounds of other metals.

As will be illustrated by the following examples, the rosins of natural origin have the beneficial effect that when used in combination with enzymes the activity of the enzymes are not substantially affected by the rosins as compared to enzymes in paint compositions prepared with synthetic binders of non-natural origin. Accordingly, it was found that no enzyme activity was present in paint compositions comprising protease and synthetic binders of non-natural origin.

The rosins are furthermore believed to have an immobilising effect on the enzymes and thus preventing the enzymes from being released from the paint composition into the environment.

The composition according to invention comprises a rosin compound wherein the content of the rosin compound is in the range of from about 5 to about 60% by weight. It is preferred that the amount of rosin compound is higher than about 10% such as up to about 20% by weight. However, it is also contemplated that the amount of rosin compound in the composition can be up to about 30%, such as up to about 40%, up to about 50% and up to about 55%. Thus, a pigmented composition according to the invention could advantageously comprise an amount of rosin compound in the range of about 10-30% by weight, and a lacquer composition could comprise up to about 60% of rosin compound by weight.

In accordance with the invention the at least one enzyme comprised in the composition according to the invention, is present in an effective amount to reduce or prevent fouling of a surface coated with the composition. In the present context the term “an effective amount” means an amount which is sufficient to at least partially reduce or prevent the settling of aquatic organisms such as bacteria, protozoa, algae and invertebrates on a surface coated with the composition according to invention. In order to test the amount of protease required in order to sufficiently reduce or prevent fouling, any type of standard or modified antifouling bioassay can be applied, including settlement assays as described by Willemsen (1994).

In a presently preferred embodiment the amount of the enzyme is in the range of about 0.1-10% by weight, including the range of about 0.2-5% by weight such as about 0.5-1% by weight.

As mentioned above, the composition according to the invention may advantageously comprise one or more enzymes. It has been found by the present inventors that by combining a protease such as a subtilisin, with amyloglucosidase and/or xylanase an additional antifouling effect was obtained Thus, it was found that the addition of amyloglucosidase and/or xylanase reduced or prevented the fouling with algae of a surface submerged in sea water. Thus, in one useful embodiment the composition according to the invention comprises an amyloglucosidase (Glucan 1,4-alpha-glucosidase; E.C. 3.2.1.3) such as AMG 300 L, Novozyme A/S, Denmark. In a further useful embodiment the composition according to the invention comprises a xylanase such as endo-1,4beta-xylanase (E.C 3.2.1.8). A useful example of such endo-1,4beta-xylanase (E.C. 3.2.1.8) is the commercially available Pulpzyme HC, Novozyme A/S, Denmark.

As it is mentioned above the composition of the present invention can advantageously be applied to prevent or reduce fouling of a surface by coating the surface with the composition. Such a surface can be any surfaces of structures that are intermittently or continuously immersed in water, such as the surfaces of vessels including boats and ships. Accordingly, in one specific embodiment of the present invention such surface is a ship hull. However, it is also contemplated that fouling of surfaces of off-shore equipment, pipes, substructures of bridges and piers, aquacultural apparatuses including fish farming nets, can be efficiently reduced or prevented

In order to improve the efficiency of the antifouling paint composition according to the invention, the composition may be combined with further biologically active agents known to suppress the settlement of marine organisms. Thus, in one embodiment the composition according to invention additionally comprises at least one algicide, herbicide, fungicide, molluscicide or other compound exhibiting anti-fouling activity.

In accordance with the invention, the antifouling paint composition can be prepared according to conventional manufacturing technology and the composition may, in addition to the protease and the rosin compound further contain components that are usual for paint compositions including binder components, pigments, fillers, dispersion agents, solvents plasticisers and other additives, and the composition can e.g. be solvent-based or water-borne.

Thus, it is contemplated that the composition of the present invention in addition to the rosin compound, which is a binder component of natural origin, can comprise one or several further synthetic binder components such as synthetic polymeric binder components including polyvinylacetate. However, it is important, which is also shown in the accompanying examples, that the further synthetic binder component is compatible with the enzyme, i.e. the enzyme is enzymatically active when in combination with the synthetic binder.

It is further contemplated that the composition according to the invention may comprise binder components such as silan compounds. Such silans may in useful embodiments be selected from silane esters, vinyl silanes, methacryloxy silanes, epoxy silanes, sulfur silanes, amino silanes, and isocyanoto silanes.

Additionally the antifouling paint composition may comprise one or more fillers, such as kaolin, silica and dolomite.

It is a further objective of the invention to provide a method for preventing fouling of a surface by an aquatic organism comprising applying to the surface an effective amount of the antifouling paint composition according to the invention. It is contemplated that aquatic organism such as those belonging to the group of bacteria, protozoa, fungi, algae and invertebrates, can be efficiently hindered in attaching to surfaces by applying the method of the present invention.

However, in one embodiment the organisms which, by the present method, can be efficiently hindered in attaching to a surface are barnacles and mussels. Such barnacles can be of the Cirripedia subclass including Balanus galeatus, Balanus amphitrite, Elminius modestus, Balanus improvisus and Balanus balanoides

As mentioned above, in a further aspect the invention relates to an antifouling paint composition which comprises at least one subtilisin (EC 3.4.21.62) said subtilisin having the following characteristica: (i) optimum activity at a pH in the range of about 7-10, and (ii) optimum activity at a temperature in the range of about 55-65° C. In one embodiment the subtilisin is Alcalase®, including Alcalase 2.5 L, Type DX®.

The invention will now be described in further details in the following, non-limiting examples.

### EXAMPLE 1

Barnacle was selected as test organism as this is an important member of the fouling community. Accordingly, mass reared cyprid larvae of the barnacle Balanus amphrite were used for settlement assays as described by Willemsen (1994).

Adult barnacles were maintained in containers with Vigorous aeration and controlled temperature (27±1° C.) and light conditions (15 hours light and 9 hours dark), and were fed on a diet of the diatom Skeletonema costatum and larvae of the brine shrimp Artemia salina. Mass-spawned nauplii were subsequently collected by pipette, transferred to 8 litre carboys and fed on Skeletonema costatum. The vessels were kept at a constant temperature of 27±1° C. and a 15/9 h light/dark photoperiod. To prevent bacterial growth antibiotics were added to the vessels (streptomycin, 36.5 mg/l, and penicillin 21.9 mg/l). The larvae reached the cyprid stage after four days. These cyprids were aged (at 5-6° C. in the dark) for five days prior to use in the settlement assays.

In order to test the efficiency of three different protease preparations an experiment was performed as described below, wherein the enzymes were tested in concentration range from 10-1000 μg/ml. The tested enzymes Alcalase, SP 234 and SP 249 were all provided by Novozyme (Novozyme A/S, Novo Allé, 2880 Bagsvaerd, Denmark). SP 234 and SP 249 are experimental preparations having a high content of protease and other non-proteolytic enzymes.

The tests were carried out in four replicates in polystyrene multi well (2×3) plates from Steriline Ltd. Between 25 and 40 cyprids were injected (using a Pasteur pipette) in the dishes containing either 2 ml of filtered seawater (controls) or enzyme solution. The test solutions were prepared by directly dissolving the enzyme solutions in 0.25 μm filtered natural seawater. The dishes were incubated for 24 hours at a temperature of 27±1° C. and with a 15:9 light-dark cycle. After incubation the cyprids were screened for signs of toxicity using a dissecting microscope. Then the test was terminated by the addition of one drop of 40% formaldehyde and the number of permanently and non-attached larvae was counted.

The results of this experiment are summarised in the below Table 1.

TABLE 1
Cyprid settlement

| Dose (μg/ml) | 10 | 50 | 100 | 500 | 1000 |
|---|---|---|---|---|---|
| Control | 80% | 80% | 80% | 80% | 80% |
| Alcalase | 50% | 0% | 0% | 0% | 0% |
| SP 234 | 85% | 88% | 75% | 10% | 0% |
| SP 249 | 50% | 70% | 40% | 20% | 20% |

As can be seen from the above Table 1, the Alcalase completely prevented barnacle settlement at 50, 100, 500 and 1000 μg/ml. The two experimental preparations did not prevent settlement as efficiently as Alcalase. It is seen that SP 234 was only able to completely prevent cyprid attachment at a relatively high concentration of 1000 μg/ml. It is also seen that SP 249 applied at a concentration of 1000 μg/l did not completely prevent cyprid settlement, as 20% of the cyprids were settled.

### EXAMPLE2

In order to further compare the enzymes applied in Example 1, an experiment based on specific enzyme activities was performed. The original enzyme samples possessed the following protease activities (HUT: Haemoglobin Units on Tyrosine basis). The HUT activity of the proteases may e.g. be determined as described in Food Chemicals CODEX, 3rd ed., (1981), pp. 496-497, National Academy Press, Washington, D.C.

| | | |
|---|---|---|
| Alcalase: | ca. 1,300,000 | HUT/g |
| SP 234: | ca. 500,000 | HUT/g |
| SP 249: | ca. 600 | HUT/g |

All enzymes were tested at a concentration corresponding to 6,000 HUT/I and 60,000 HUT/I, and the settlement assays were performed as previously described in Example 1. The results from this test are shown in the below Table 2.

TABLE 2

| | | ppm | % Cyprid | |
|---|---|---|---|---|
| Treatment | HUT/I | (pg/ml) | settlement | G-test (ns = not significant |
| Control | 0 | 0 | 63 | — |
| Alcalase | 6,000 | 4.6 | 34 | 19.37; p < 0.005 |
| | 60,000 | 46 | 0.8 | 124.1: p << 0.005 |
| SP 234 | 6,000 | 12 | 62 | 0.005; ns |
| | 60,000 | 120 | 52 | 2.665; ns |
| SP 249 | 6,000 | 1,000 | 52 | 2.702; ns |
| | 60,000 | 10,000 | 0 | 134.6; p << 0.005 |

It is clearly seen from the above Table 2 that Alcalase significantly inhibits settlement at 6,000 HUT/I (4.6 ppm) and completely prevents settlement at 60,000 HUT/I (46 ppm). SP 234 has no significant influence on settlement at both 6,000 HUT/I and 60,000 HUT/I. SP 249 completely inhibits settlement at 60,000 HUT/I but has no significant influence at 6,000 HUT/I.

In all solutions, except SP 249 at 6,000 HUT/I, cyprids looked healthy after 24 hours of incubation, indicating the non-toxic character of the solutions. In SP 249 larvae were still alive in the 6,000 HUT/I solution, but they did not show normal swimming and settlement behaviour.

### EXAMPLE 3

Based on the above settlement experiments Alcalase was chosen as a candidate for further studies. In order to test the Alcalase enzyme activity in individual and typical paint binder components, the below experiment were performed. Accordingly, Alcalase (Alcalase 2.5 L Type DX®, Novozyme) was tested for its compatibility with 7 different typical binders commonly used in antifouling paints, by testing the residual enzymatic activity after 24 hours of incubation at 36° C.

The seven different binders were: modified rosin, hydrogenated rosin, polyvinyl acetate emulsion, polyvinyl methyl ether, polyvinyl chloride copolymer, acrylic resin copolymer, and silicone binder. The above tested binders were all obtained from Hempel Marine Paints A/S (Hempel Marine Paints A/S, Lundtoftevej 150, 2800 Lyngby, Denmark).

Alcalase was added to and mixed with the above binders at four different enzyme concentrations (0.25%, 0.50%, 1.0%, and 2%, by weight). The amount of added enzyme was based on the dry matter content of the different binders. Small drops of the different binder samples containing the Alcalase were made and allowed to dry. In order to obtain a sufficiently thick layer of the drops, additional drops were applied onto the dried drops of the enzyme/binder mixture. The weight of the dried drops were approximately in the range of 0.1-0.15, g per drop.

The dried drops of the enzyme/binder mixture containing different amounts of enzyme were, together with a control without enzyme, incubated on an skim milk agar plate at 36° C. for 20 days.

TABEL 3
Enzyme activity (clear zone, visually detected)

| Enzyme amount (%) | 0.25 | 0.5 | 1.0 | 2 |
|---|---|---|---|---|
| Modified rosin, dry matter 55% | + | + | + | + |
| Hydrogenated rosin, dry matter 50% | + | + | + | + |
| Polyvinyl acetate emulsion, dry matter 55% | − | − | + | + |
| Polyvinyl methyl ether, dry matter 35% | − | − | − | − |
| Polyvinyl chloride copolymer, dry matter 40% | − | − | − | − |
| Acrylic resin, copolymer, dry matter 40% | − | − | − | − |
| Silicone binder, dry matter 63% | − | − | − | − |

It is clearly seen from the above Table 3, that the protease activity of Alcalase 2.5 L Type DX® was highly influenced by the binder type. Thus, it can be seen that the protease was active when in combination with rosin types of natural origin, namely modified rosin and hydrogenated rosin. In contrast hereto, it can be seen that no protease activity was detected when the Alcalase was combined with the synthetic binders of non-natural origin, namely polyvinyl methyl ether, polyvinyl chloride copolymer, acrylic rosin copolymer and silicone binder.

Thus, it can be concluded that the protease of Alcalase 2.5 L Type DX® can be highly efficient for the purpose of antifouling agent in a marine paint having rosin types of natural origin.

### EXAMPLE 4

Field experiments were performed in seawater in order to test the efficiency of a paint composition comprising Alcalase 2.5 L Type DX® in combination with two other commercially available enzyme preparations (amyloglucosidase and xylanase preparation). Accordingly, two paints containing enzymes were prepared. The paints were named BioB and BioS depending on whether they were solvent-based (BioB) or water-based (BioS).

The solvent-based paint (BioB) contained the following components; Natural rosin hydrogenated (20 wt %), acryl resin (20 wt %), dispersion agent (0.75 wt %), titandioxid, dolomit (10 wt %), talcum powder (1.25 wt %), aromatic hydrocarbon (3 wt %) and polyvinylmethylether 5.0 wt %).

The water-based paint (BioS) contained the following components; Polyvinylacetate (13 wt %), dispersion agent (0.75 wt %), titandioxid (10.0 wt %), dolomit (40.0 wt %), talcum powder (1.25 wt %), natural rosin (13.0 wt %) and water (11.0 wt %).

The following enzymes were applied:

Alcalase: (Alcalase 2.5 L Type DX®, Novozyme)

AMG: (AMG 300 L, Novozymes A/S, Denmark)

Pulpzyme: (Pulpzyme HC, Novozymes A/S, Denmark)

The enzymes were added to the two different marine paints in the different amounts given in table 4 A.

TABLE 4A

| | | Totel enzyme conc. |
|---|---|---|
| Paint | Enzyme (% weight) | (% weight) |
| A 0.5 | Alcalase (0.5%) | 0.5 |
| A 2.0 | Alacalase (2.0%) | 2.0 |
| B 0.5 | AMG (0.5%) | 0.5 |
| B 2.0 | AMG (2.0%) | 2.0 |
| C 0.5 | Pulpzyme (0.5%) | 0.5 |
| C 2.0 | Pulpzyme (2.0%) | 2.0 |
| D 2.0 | Alcalase (1%) + AMG (1%) | 2.0 |
| E 2.0 | Alcalase (1%) + Pulpzyme (1%) | 2.0 |
| F 2.0 | Alcalase (2/3%) + AMG (2/3%) + | 2.0 |
| | Pulpzyme (2/3%) | |

Sand-blasted acrylic plates (10×20×0.5 cm) were painted with one of the two marine paints with a surface layer of approximately 130 cm 2 and with a film thickness of 100 micron for BioB and 85 micron for BioS, respectively

After drying, the panels were mounted on a raft with 5×3 panels. The rafts were immersed into seawater in two different harbours in Denmark (Jyllinge with stagnant water and Ellsinore with high water replacement) for six month (5 May 2000 to 13 Nov. 2000). The rafts were inspected monthly.

The rafts were immersed in such a way that the upper part of the panel was approximately 1 meter below the water surface.

At the end of the period the panels were taken to the laboratory and evaluated for number of barnacles attached. The flora fouling was also evaluated. The surfaces of the painted panels were also evaluated for-structural changes (cracks and holes).

The results from the experiment performed at Ellsinore can be seen from the below table 4 B.

TABLE 4B

| | | Number of |
|---|---|---|
| Panel number | Explanation to panel number | barnacles |
| Blank | Panel without paint | 66 |
| 0-0 | BioB without enzymes | 17 |
| D 2.0 | BioB + Alcalase + AMG | 4 |
| E 2.0 | BioB + Alcalase + Pulpzyme | 9 |
| F 2.0 | BioB + Alcalase + AMG + Pulpzyme | 7 |
| W-0-0 | BioS without enzymes | 38 |
| W-D 2.0 | BioS + Alcalase + AMG | 30 |
| W-E 2.0 | BioS + Alcalase + Pulpzyme | 45 |
| W-F 2.0 | BioS + Alcalase + AMG + Pulpzyme | 44 |
| Ref. 1 | Bravo, Hempel A/S | 0 |
| Ref. 2 | Seatech, Hempel A/S | 0 |

It is clearly seen from the above Table 4B, that on the panels painted with BioB comprising enzymes, only a very few barnacles were attached as compared to the panels painted with BioS without enzymes. Thus, it can be seen that the combination of BioB+Alcalase+AMG results in a significant reduction of the number of barnacles attached (no of barnacles 4) as compared to BioB without enzymes (no. of barnacles 17). Accordingly, the combination of BioB+Alcalase+AMG resulted in an almost complete inhibition of the attachment of barnacles. In comparison, the two commercial antifouling products containing the biocides Irgarol and Diuron completely inhibited the attachment of barnacles.

Selected samples from the BioB and BioS panels were inspected using a magnifying glass (4×) and the fouling did not contain any other animals than barnacles on the panels painted with paint containing enzymes. Regarding the flora, BioB panels with enzymes only had a few types of algae attached with the siliceous algae Schizonema as the dominant, whereas the control was completely covered with algae. The algae fouling on the BioB panels with enzymes was later easily removed from the panels with a wet sponge. Accordingly, the use of Alcalase in combination with AMG and/or Pulpzyme significantly reduced the algae fouling.

BioB and BioS panels were inspected for cracks and holes with a magnifying glass (4×). The surfaces of the BioB panels were still fully intact after six months in seawater. No cracks and holes could be detected. However, BioS panels showed some cracks and holes where the fouling could be detected.

### REFERENCES

Willemsen P. R. Antifoulants from marine invertebrates—Sponges. In: Proceedings Workshop “Biofouling: problems and solutions” University of New South Wales, Syney, Australia, 13-14Apr. 1994.

1. An antifouling paint composition which comprises at least one proteolytically active enzyme and at least one rosin compound, wherein the at least one proteolytically active enzyme is present in an effective amount to reduce or prevent fouling of a surface coated with said composition.

2. The composition according to claim 1, wherein the composition further comprises at least one hemicellulolytically active enzyme.

3. The composition according to any of claims 1 and 2, wherein the composition further comprises at least one amylolytically active enzyme.

4. The composition according to claim 1, wherein the composition further comprises at least one cellulolytically active enzyme.

5. The composition according to any of claims 2 and 3, wherein the at least one proteolytically active enzyme is a protease.

6. The composition according to claim 5, wherein the proteolytically active enzyme is an endopeptidase.

7. The composition according to claim 6, wherein the endopeptidase is a Subtilisin (EC 3.4.21.62).

8. The composition according to claim 7 wherein the Subtilisin (EC 3.4.21.62) has the following characteristica: (i) optimum activity at a pH in the range of about 7-10, and (ii) optimum activity at a temperature in the range of about 55-65° C.

9. The composition according to claim 8 wherein the Subtilisin (EC 3.4.21.62) is Alcalase®.

10. The composition according to claim 5, wherein the at least one hemicellulolytically active enzyme is selected from the group consisting of Endo-1,4-beta-xylanase (E.C. 3.2.1 8), Xylan endo-1,3-beta-xylosidase (E.C. 3.2.1.32). Glucuronoarabinoxylan endo-1,4-beta-xylanase (E.C. 3.2.1.136), Beta-mannosidase (E.G. 3.2.1.25), Mannan endo-1,-beta-mannosidase (E.C. 3.2.1.78) and Mannan endo-1,6beta-mannosidase (E.C. 3.2.1.101).

11. The composition according to claim 10, wherein the at least one hemicellulolytically active enzyme is a xylanase.

12. The composition according to claim 11, wherein the xylanase is an endo-1,4-beta-xylanase (E.C. 3.2.1.8).

13. The composition according to claim 5, wherein the at least one amylolytically active enzyme is an amylase.

14. The composition according to claim 5, wherein the at least one amylolytically active enzyme is selected from the group consisting of α- and β-amylases, amyloglucosidases (E.C. 3.2.1.3), pullulanases, α-1,6-endoglucanases, α-1,4-exoglucanases and isoamylases.

15. The composition according to claim 5, wherein the at least one amylolytically active enzyme is an amyloglucosidase.

16. The composition according to claim 15, wherein the amyloglucosidase is an 1,4alpha-glucosidase.

17. The composition according to claim 5, wherein the at least one hemicellulolytically active enzyme is a xylanase and wherein the at least one amylolytically active enzyme is an amyloglucosidase.

18. The composition according to claim 17, wherein the xylanase is an endo-1,4-beta-xylanase (E.C. 3.2.1.8) and wherein the amyloglucosidase is 1,4-alpha-glucosidase (E.C. 3.2.1.3).

19. The composition according to claim 1 wherein the rosin compound is selected from the group consisting of a rosin, a rosin derivative and a rosin metal salt.

20. The composition according to claim 19 wherein the rosin is selected from the group consisting of a tall rosin, a gum rosin and a wood rosin.

21. The composition according to claim 19 wherein the rosin derivative is selected from the group consisting of a hydrogenated rosin, a modified rosin obtained by reacting rosin with maleic anhydride, a formylated rosin, and a polymerised rosin.

22. The composition according to claim 19 wherein the rosin metal salt is selected from the group consisting of a zinc rosinate, a calcium rosinate, a copper rosinate, and a magnesium rosinate.

23. The composition according to claim 1 wherein the content of the rosin compound is in the range of about 5-60% by weight.

24. The composition according to claim 1 wherein the amount of the at least one proteolytically active enzyme is in the range of about 0.1-10% by weight.

25. The composition according to claim 24 wherein the amount of the at least one proteolytically active enzyme is in the range of about 0.2-5% by weight.

26. The composition according to claim 24 wherein the amount of the at least one proteolytically active enzyme is in the range of about 0.5-1% by weight.

27. A composition according to claim 1 wherein the surface is a surface that is at least occasionally immersed in water, wherein said water includes fresh, salt or brackish water.

28. A composition according to claim 27 wherein the surface is selected from the group consisting of the surfaces of vessels including boats and ships, ship hulls, off-shore equipment, pipes, substructures of bridges, piers and aquacultural apparatuses including fish farming nets.

29. A composition according to claim 1, further comprising at least one algicide, herbicide, fungicide, molluscicide or other compound exhibiting anti-fouling activity.

30. A composition according to claim 1 further comprising a binder component, suitable for marine applications and a pigment.

31. A method for preventing fouling of a surface by an aquatic organism, said method comprising applying to the surface an effective amount of an antifouling paint composition according to any of claims 1 to 30.

32. A method according to claim 31 wherein the aquatic organism is selected from the group consisting of bacteria, protozoa, fungus, algae and invertebrates.

33. A method according to claim 32 wherein the aquatic organism is selected from barnacles and mussels.

34. A method according to claim 32 wherein the aquatic organism are of the Cirripedia subclass including Balanus galeatus, Balanus amphitrite, Elminius modestus, Balanus improvisus and Balanus balanoides.

35. An antifouling paint composition which comprises at least one subtilisin (EC3.4.21.62), said subtilisin having the following characteristica: (i) optimum activity at a pH in the range of about 7-10, and (ii) optimum activity at a temperature in the range of about 55-65° C.

36. An antifouling paint composition according to claim 28 wherein the subtilisin is Alcalase®.

37. An antifouling paint composition according to claim 29 wherein the Alcalase® is Alcalase 2.5 L, Type DX®.

38. Use of subtilisin (EC 3.4.21.62) in an antifouling paint composition said subtilisin having the following characteristics: (i) optimum activity at pH in the range of about 7-10, and (ii) optimum activity at a temperature in the range of about 55-65° C.

39. Use according to claim 31 wherein the subtilisin is Alcalase®.

40. Use according to claim 32 wherein the Alcalase® enzyme composition is Alcalase 2.5 L, Type DX®.
